(19) United States
(12) Patent Application Publication
(10) Pub. No.: US 2017/0129933 A1
(43) Pub. Date: May 11, 2017

(54) PURIFICATION OF TGF-BETA SUPERFAMILY PROTEINS

(71) Applicants: BIOVENTUS, LLC, Durham, NC (US); FUJIFILM DIOSYNTH BIOTECHNOLOGIES U.S.A., LLC, Morrisville, NC (US)

(72) Inventors: Christopher T. Brown, Chelmsford, MA (US); Patrick D. Robertson, Morrisville, NC (US); Eugene Kingsley, Morrisville, NC (US); Anthony Caronna, Morrisville, NC (US)

(73) Assignees: BIOVENTUS, LLC, Durham, NC (US); FUJIFILM DIOSYNTH BIOTECHNOLOGIES U.S.A., LLC, Morrisville, NC (US)

(21) Appl. No.: 15/331,137

(22) Filed: Oct. 21, 2016

Related U.S. Application Data
(60) Provisional application No. 62/245,727, filed on Oct. 23, 2015.

(51) Int. Cl. C07K 14/51; B01D 15/32; B01D 15/42; C07K 1/20; B01D 15/38

(52) U.S. Cl. CPC C07K 14/51; C07K 1/20; B01D 15/3804; B01D 15/424; B01D 15/327

(57) ABSTRACT

Methods of purifying TGF-β superfamily proteins, including osteogenic proteins such as bone morphogenetic proteins (BMPs), are disclosed.

### CROSS REFERENCE TO RELATED APPLICATION

This application claims the benefit of U.S. Provisional Application No. 62/245,727, filed Oct. 23, 2015, which is hereby incorporated by reference in its entirety.

### FIELD

The present disclosure relates generally to protein purification methods for the transforming growth factor-β (TGF-β) superfamily of proteins. Specifically, the present disclosure relates to methods of purification of such proteins, including bone morphogenetic proteins (BMPs).

### BACKGROUND

The transforming growth factor beta (TGF-β) superfamily of proteins is a large family of multifunctional proteins that regulate a variety of cellular functions, including cellular proliferation, migration, differentiation and apoptosis. Bone morphogenetic proteins (BMPs) comprise a subfamily within the TGF-β superfamily member that serve as signal transduction ligands that regulate, among other things, bone, cartilage and connective tissue growth. High levels of recombinant BMPs may be produced in cell cultures (e.g., yeast, E. coli and mammalian cells) using cells transformed with an expression vector containing the corresponding DNA. The BMPs secreted from the host cell must be isolated and purified from the host cell culture medium. Typically, the culture media contains nutrients (e.g., vitamins, amino acids, co-factors, minerals etc.), growth factors/supplements, various other host cell substances (e.g., nucleic acids, membrane components etc.) and additional unwanted host cell proteins. The cell culture media may also contain a variety of undesirable BMP gene products, including product forms lacking post-transitional modifications and proteolytically-degraded forms of BMP that closely resemble the full-length product of interest.

Generally, BMPs have been difficult to purify due to their insolubility in conventional buffer systems and their tendency to aggregate and precipitate at physiological pH (Ruppert et al., 1996 Eur. J. Biochem. 1996, 237(1):295-302). Indeed, Steckert et al. (Therapeutic Proteins, Methods in Molecular Biology, Volume 308, 2005, pp 301-318) has demonstrated that rhBMP-2 will reversibly self-associate as a function of pH and salt. BMP purifications typically rely on the use of chaotropes and other strong protein denaturants such as detergents and water soluble organics to maintain protein solubility during the purification process.

Historically, heparin and heparin-like affinity resins have been utilized for the capture and purification of BMPs. For example, the N-terminal sequence of BMP-2, which contains 10 basic residues, is a known heparin binding site (Ruppert et al 1996). The non-covalent reversible interaction between heparin and BMPs ensure that binding occurs with minimal impact on growth factor structure and function. Traditional heparin resins are limited by their inability to be treated with high concentrations of NaOH, the standard sanitization method, and safety issues associated with the potential for leaching of the heparin ligand. Alternatives to heparin resins include Cellufine Sulfate (JNC Corporation), a resin functionalized with sulfate esters on a backbone of cellulose, which in some instances function as a heparin analog for purification of heparin binding proteins. Due to Cellufine Sulfate's low (3 kDa) exclusion limit, large molecules only adsorb to the exterior of the beads, resulting in limited capacity as the ligands residing in the interior of the beads are not accessible.

Conventional hydrophobic interaction chromatography (HIC) separates molecules based on the adsorption of protein through non-covalent interactions between hydrophobic regions on the protein surface and hydrophobic groups (e.g., phenyl, octyl or butyl) chemically attached to the resin. In a typical HIC process, protein solutions are applied to the medium in a high-salt buffer which decreases solvation and exposes hydrophobic regions on the protein molecules to promote interaction with the hydrophobic ligands on the medium. The more hydrophobic the molecule, the less salt is needed to promote binding and typically proteins are eluted from the medium by decreasing the salt concentration. Most often, a gradient of decreasing salt concentration is applied to the HIC medium to elute samples from the medium in order of increasing hydrophobicity. Sample elution may also be assisted by the addition of water soluble organic modifiers or detergents to the elution buffer.

### SUMMARY

In one aspect, the present disclosure relates to a method of purifying protein(s) of the TGF-β family of proteins, including, for example, a bone morphogenetic protein (BMP) from a fluid (for instance, a cell culture supernatant, a bodily fluid or any other fluid comprising such protein), comprising the steps of: contacting the fluid comprising the BMP with a hydrophobic interaction chromatography medium under conditions in which the BMP is soluble within the fluid, wherein the fluid includes at least one salt at a concentration above a predetermined threshold, thereby facilitating an association of the BMP with the hydrophobic interaction chromatography medium; contacting the hydrophobic interaction chromatography medium with a first mobile phase comprising a first agent that promotes the solubility of the BMP, the first mobile phase having a salt concentration similar to a salt concentration of the initial fluid; contacting the hydrophobic interaction chromatography medium with a second mobile phase lacking the first agent that promotes the solubility of the BMP, thereby increasing an association between the BMP with the chromatography medium; contacting the hydrophobic interaction chromatography medium with a third mobile phase having a dissimilar salt concentration relative to one or more of the fluid, the first mobile phase and the second mobile phase, thereby decreasing an association of a second, non-BMP with the hydrophobic interaction chromatography medium; and eluting the BMP from the hydrophobic interaction chromatography medium by contacting the hydrophobic interaction chromatography medium with an elution mobile phase comprising a second agent, different from the first agent, that promotes the solubility of the BMP and disrupts the association with the hydrophobic interaction chromatography media. The concentration of the second agent may be varied over time. Alternatively, the concentration of the second agent may be constant over time. The hydrophobic interaction chromatography medium may not be functionalized with a peptide affinity ligand. The first agent that promotes the solubility of the BMP may be urea. The urea may be present in the first mobile phase at a concentration of 5-8M. The first mobile phase may include 50 mM glycine and 2M sodium chloride. The urea may be present in the fluid at a concentration of at least 3M. The fluid may include at least 1M sodium chloride. The second agent that promotes the solubility of the BMP may be hexylene glycol. The fluid may include an eluent from an ion exchange chromatography medium. The product BMP yield may be at least 60%. The purity of the BMP may be at least 90%.

In another aspect, the present disclosure relates to a method of purifying protein(s) of the TGF-β family of proteins, including, for example, a bone morphogenetic protein (BMP) from a sample, comprising the steps of: loading an affinity-like chromatography medium with a solution containing BMP under conditions such that at least a portion of the BMP binds to the affinity-like chromatography medium; eluting at least a portion of the BMP from the affinity-like chromatography medium; loading a hydrophobic interaction chromatography medium with the BMP-containing eluent from affinity-like chromatography medium under conditions such that at least a portion of the BMP binds to the hydrophobic interaction chromatography medium; eluting at least a portion of the BMP from the hydrophobic interaction chromatography medium; loading a cation exchange medium with the BMP-containing eluent from the hydrophobic interaction chromatography medium under conditions such that at least a portion of the BMP binds to the cation exchange medium; eluting at least a portion of the BMP from the cation exchange medium; and concentrating the BMP in a suitable buffer.

In another aspect, the present disclosure relates to a method purifying protein(s) of the TGF-β family of proteins, including, for example, a bone morphogenetic protein (BMP) from a fluid, comprising the steps of: loading the fluid containing BMP onto a hydrophobic interaction medium, wherein the fluid includes urea and a first salt at a first concentration, and wherein the BMP is in solution in the fluid; washing the hydrophobic interaction medium with a first solution, wherein a concentration of the salt in the first solution is less than the first concentration, the first solution does not include urea, and the BMP is less soluble in the first solution than in the fluid; and eluting the BMP with a second solution that does not include the first salt or urea. The urea may be present in the fluid at a concentration of at least 3M. The fluid may include at least 1M sodium chloride. The second solution may promote the solubility of the BMP. The second solution may include hexylene glycol.

In another aspect, the present disclosure relates to a method of purifying protein(s) of the TGF-β family of proteins, including, for example, a bone morphogenetic protein (BMP) from a fluid a first solution, comprising the steps of: contacting a hydrophobic interaction chromatography medium with the first solution, wherein the first solution is characterized by a first solubility of the BMP therein; contacting the hydrophobic interaction chromatography medium with a second solution characterized by a second solubility of the BMP that is less than the first solubility; and contacting the hydrophobic interaction chromatography medium with a third solution characterized by a third solubility of the BMP that is greater than the second solubility.

### BRIEF DESCRIPTION OF THE DRAWINGS

Certain embodiments of the present invention are illustrated by the accompanying figures. It will be understood that the figures are not necessarily drawn to scale and that details not necessary for an understanding of the invention or that render other details difficult to perceive may be omitted. It will be understood that the invention is not necessarily limited to the particular embodiments illustrated herein.

FIG. 1 provides an overview of the BMP purification process, according to one embodiment of the present disclosure.

FIG. 2 depicts a chromatogram from an affinity-like chromatography purification, according to one embodiment of the present disclosure.

FIG. 3 depicts a chromatogram of a hydrophobic interaction chromatography purification, according to another embodiment of the present disclosure.

FIG. 4 depicts a chromatogram of a cation exchange chromatography purification, according to yet another embodiment of the present disclosure.

### DETAILED DESCRIPTION OF THE PREFERRED EMBODIMENTS

Expression of BMP may be achieved by inserting a suitable gene into an expression vector, transforming a suitable mammalian cell with the expression vector and selecting for cells which express the BMP. A variety of mammalian cell lines may be used to express BMP, such as CHO (Chinese Hamster Ovary), COS, BHK, Balb/c 3T3, 293 and similar cell lines known in the art. These cells may be grown in any suitable culture medium known in the art.

The following examples describe the isolation and purification from cell culture media of a designer BMP, as described in U.S. Pat. No. 8,952,131, which is incorporated by reference in its entirety. It will be understood, however, that the present disclosure may be used with similar results for other protein members of the TGF-β family of proteins, particularly the bone morphogenetic proteins, including BMP-1 through BMP-15 and recombinant, homodimeric, heterodimeric, mutant and/or chimeric versions thereof. The steps outlined below are for illustrative purposes only, and are not intended to limit the scope of the present disclosure.

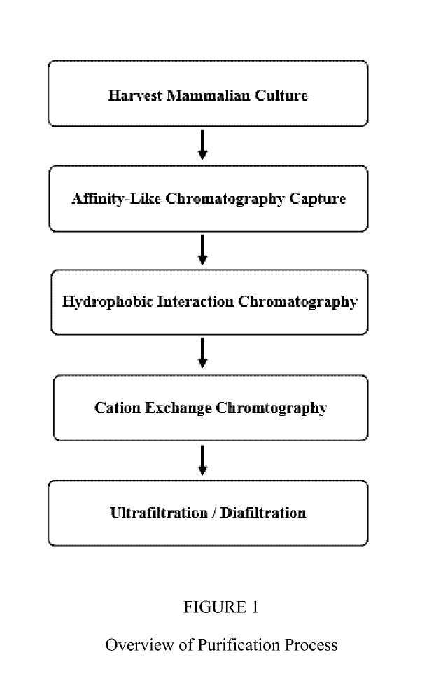

FIG. 1 provides an overview of the purification process of the present disclosure. While the order of the steps set forth includes a preferred embodiment, it should be appreciated that numerous variations and modifications are within the scope of the present disclosure. For example, the order of steps may be re-configured if desired and steps may be omitted.

### Harvesting Step

Upon secretion from a suitable cellular expression system, the positively charged BMP protein tends to bind tightly to the negatively charged outer surface of the host cell. Dextran sulfate may be added to the culture medium to disrupt this binding without damaging the BMP and/or disturbing the host cell such that additional unwanted cellular components are released into the media. The culture medium is then separated from the cultured cells after pH adjustment to pH 6.7 with 5% v/v 1.1M 4-morpholineethanesulfonic acid-(2-[N-morpholino] ethanesulfonic acid) (MES) by depth filtration or, alternatively, centrifugation.

### Affinity-Like Capture (Step-1)

In the first purification step the culture medium is loaded onto an “affinity-like” medium to remove the dextran sulfate, clear host cell proteins and other unwanted residual products and concentrate the BMP. As described in U.S. Patent Application No. 20030036629, hereby incorporated by reference in its entirety, Matrex Cellufine® Sulfate (JNC Corporation) may be used as an affinity-like medium for the initial purification of BMPs such as recombinant human BMP-2 (rhBMP-2) from conditioned cell culture media. Cellufine Sulfate includes a resin composed of spherical cellulose beads functionalized with dextran sulfate, which simultaneously acts as an ion-exchange medium and a heparin analog that can bind heparin binding sites in target proteins including BMPs. Such multi-functional media (referred to hereinafter as “affinity-like” or “pseudo-affinity”) can also compete with dextran sulfate present within the culture media for binding to BMPs at appropriate pH values (including, without limitation, pH 6.7). Following appropriate washing, the bound BMP may be eluted using 0.5M L-arginine added to 50 mM TRIS plus 0.5M NaCl.

Although Cellufine Sulfate may be used to effectively capture the BMP from the conditioned cell culture medium, it requires storage in highly flammable ethanol, cannot be sterilized with NaOH having a concentration above 0.1M, and has relatively low pressure tolerances. The affinity-like capture step of the present disclosure preferably utilizes a robust Capto™ DeVirS resin (GE Healthcare, Marlborough, Mass.) or like media with affinity-like functionality. Capto™ DeVirS, which includes dextran sulfate linked to a highly cross-linked agarose base matrix, offers distinct advantages over either heparin or Cellufine resins, including increased alkali stability, no need for ethanol storage, higher flow rate (600 cm/hr vs 150 cm/hr) and higher capacity (6 mg/ml vs 0.4 mg/ml).

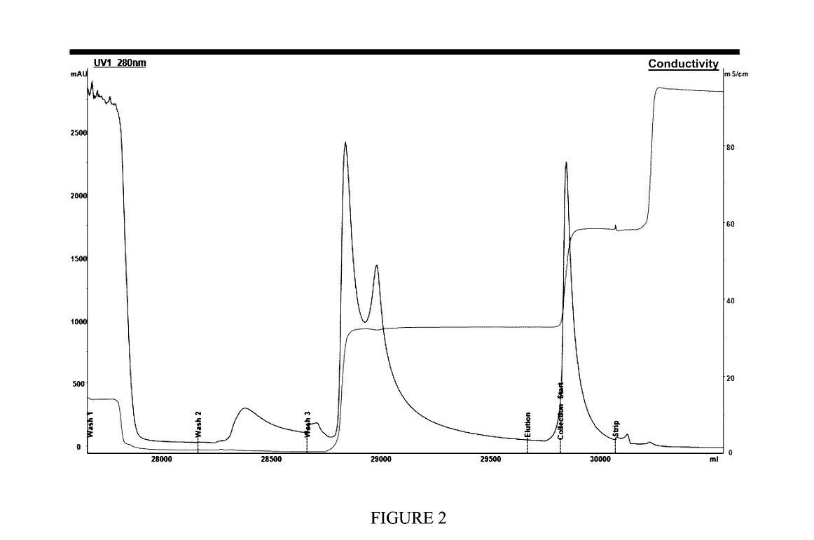

Referring to FIG. 2, in an exemplary affinity-like purification step, titrated media from the harvesting step may be loaded onto an equilibrated medium at a linear flow rate of ≦10 cm/min. The medium is then washed three times. The first wash may include one volume of 50 mM (YMS), pH 5.6; the second wash may include one volume of 50 mM MES, 6M urea, pH 5.6; and the third wash may include one volume of 50 mM MES, 6M urea, 0.5M NaCl, pH 5.6. Isocratic elution of the medium may then be performed using 50 mM MES, 6M urea, 1M NaCl, pH 5.6. The eluent is then acidified to 3% v/v acetic acid. Surprisingly, the BMP yield following the elution step is 88.4% with a purity verified by reverse phase chromatography of 60-75%.

### Hydrophobic Interaction Chromatography (Step-2)

Hydrophobic Interaction Chromatography (HIC) is based on the reversible interaction between a protein and the hydrophobic ligand bound to the chromatography matrix. Most proteins, and to a lesser extent hydrophilic molecules (e.g., DNA and carbohydrates), include hydrophobic regions on or near their surface. In the presence of high salt concentrations and/or high ionic strength buffers the interaction between hydrophobic regions of the protein and corresponding hydrophobic areas on the solid support is enhanced. Unlike most elution procedures which involve incrementally increasing the salt concentration, elution from a HIC medium involves decreasing the salt concentration such that the hydrophobic interaction is reversed and the protein de-sorbs from the medium. HIC is therefore an excellent purification step following high salt isocratic elution from the affinity-like medium.

While amenable to purification using conventional HIC purification methodology, the unusual solubility properties of BMP family members, and their propensity to reversibly aggregate and precipitate, provide an opportunity to perform HIC purifications in a “mixed-mode” which utilizes multiple interactions to achieve separation. Unlike conventional protocols, the HIC step of the present disclosure simultaneously exploits the conventional interaction between the BMP molecules and the HIC resin in addition to secondary interactions driven by the inherent solubility properties of BMP molecules themselves. While not wishing to be bound by any theory, it is believed that the solubility properties of BMP molecules can be manipulated while bound to the HIC medium in a manner which increases the effectiveness of a HIC purification process by utilizing agents that either promote or inhibit solubility. U.S. Pat. No. 7,754,689 (the “'689 patent,” hereby incorporated by reference in its entirety) describes a purification method which exploits the self-association property of BMPs using specialty resins comprised of BMP or BMP-derived peptides covalently immobilized on chromatography media. As described herein, these BMP-derived specialty resins are not necessarily required, and similar principles can be applied using standard commercially available HIC resins. An additional distinction over the '689 patent is that the HIC step of the present disclosure binds the BMP to the resin in the soluble state, whereas the '689 patent requires the BMP to be loaded under conditions that promote aggregation and precipitation which may result in poor recovery. To achieve binding, BMPs are loaded in solutions containing high concentrations of salt (e.g., 1-2M NaCl) and an agent that promotes BMP solubility (e.g., 6-8M urea). The first wash contains the same concentration of salt and solubilizing agent to flush residual loading solution and wash off any unbound contaminants. After the initial washing, the solubilizing agent is removed by a second wash to transition the BMP to the insoluble phase to allow the BMP to remain bound to the medium in the absence of salt. To achieve this, the second wash contains the same concentration of salt, but the solubilizing agent is reduced or eliminated altogether. The medium is subsequently washed with a solution devoid of salt to release freely soluble non-BMP contaminants from the medium. The bound BMP is then eluted from the medium using a solution that returns the aggregated BMP to the soluble phase (e.g., 20-50% hexylene diol).

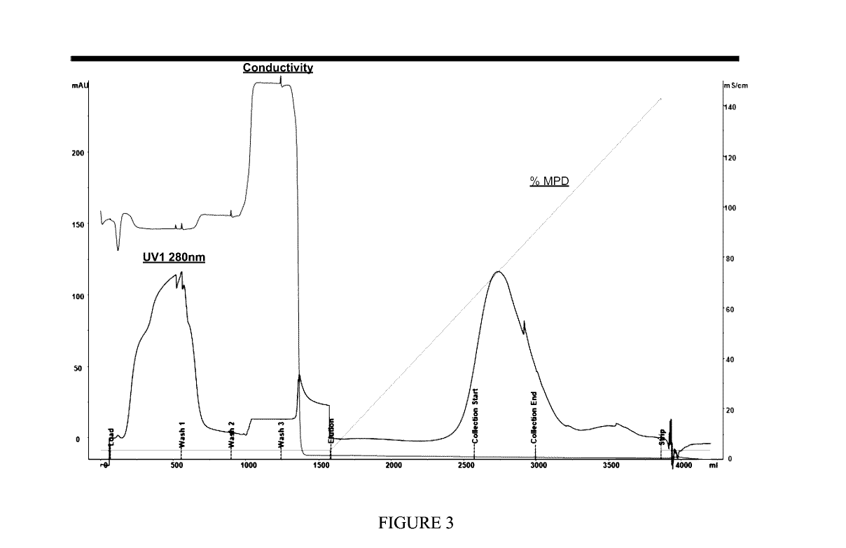

Referring to FIG. 3, in an exemplary HIC purification step, the acidified eluent from the affinity-like capture step may be diluted 1:1 with 3M NaCl, 6M urea, sterile filtered and loaded onto a Phenyl 6FF (GE Healthcare) HIC medium. Impurities such as host cell proteins and other residual contaminants (e.g., host cell proteins, cell culture media components, DNA etc.) may be washed off the medium while the BMP remains bound. The first wash may include one volume of 50 mM glycine, 2M NaCl, 6M urea, pH 3.0; the second wash may include 50 mM glycine, 2M NaCl, pH 3.0; and the third wash may include 50 mM glycine, pH 3.0. Gradient elution of the medium may then be performed using 50 mM glycine, 50% hexylene glycol (2-Methyl-2,4-pentanediol, MPD), pH 3.0 (20 column volumes, (CV)). The eluent is then sterile filtered before proceeding to the cation exchange polishing step. The BMP yield following the HIC column step is 60-75%, with a purity verified by reverse phase chromatography of 93-99%. Thus, the present disclosure provides BMP yields that are approximately the same as conventional HIC protocols but with a purity (as measured by reverse phase chromatography) that is significantly higher than conventional HIC protocols.

In another embodiment, a hybrid version of the “mixed-mode” approach may be performed which includes the same initial loading and washing steps as described above, but allows protein elution to be performed by manipulating salt concentrations according to conventional HIC protocols. Briefly, the BMPs are loaded in a solution containing high concentrations of salt (e.g., 1-2M NaCl) and an agent that promotes BMP solubility (e.g., 6-8M urea). The first wash contains the same concentration of salt and solubilizing agent to flush residual loading solution and wash off any unbound contaminants. After the initial washing, the solubilizing agent is removed by a second wash (e.g., containing the same concentration of salt, but is absent the solubilizing agent) to transition the BMP to the insoluble phase such that the BMP remains bound to the medium in the absence of salt. The medium is subsequently washed with a solution devoid of salt to release freely soluble non-BMP contaminants from the medium. BMP is then returned to soluble state while remaining bound to the media by two sequential washes that restore the initial salt concentration with subsequent reintroduction of the solubilizing agent. The bound BMP is then eluted from the medium by decreasing the salt concentration in either gradient or step-wise manner according to conventional HIC protocols, permitting users to incorporate a “mixed-mode HIC” purification into a purification process designed around a standard HIC protocol.

By way of example, in the hybrid version of the “mixed-mode” HIC purification approach the acidified eluent from the affinity-like capture step may be diluted 1:1 with 3M NaCl, 6M urea, sterile filtered and loaded onto a Phenyl 6FF (GE Healthcare) HIC medium. Impurities such as host cell proteins and other residual contaminants (e.g., host cell proteins, cell culture media components, DNA etc.) may be washed off the medium while the BMP remains bound. The first wash may include one volume of 50 mM glycine, 2M NaCl, 6M urea, pH 3.0; the second wash may include 50 mM glycine, 2M NaCl, pH 3.0; the third wash may include 50 mM glycine, pH 3.0; the fourth wash may be similar to the second wash and include 50 mM glycine, 2M NaCl, pH 3.0; the fifth wash may be similar to the first wash and include 50 mM glycine, 2M NaCl, 6M urea, pH 3.0. Elution may be achieve by transitioning the media to 50 mM glycine, 6M urea, pH 3.0 in either gradient or step-wise manner.

It should be appreciated that a variety of salts other than NaCl may be compatible with the HIC resin by promoting hydrophobic interactions. Non-limiting examples of salts that may be used to enhance the availability of hydrophobic portions of the BMP to the HIC resin may include Na₂SO₄, K₂SO₄, (NH₄)₂SO₄, Na₂HPO₄, KCl, LiCL, KSCN and CH₃COONH₄. It should also be appreciated that a variety of solubilizing agents other than hexylene glycol (MPD) may be used to elute BMP from the hydrophobic interaction medium. Non-limiting examples of suitable solubilizing agents may include chaotropic agents (e.g., urea, arginine, guanidine-HCl), detergents (e.g. CHAPS, Triton-X100, Polysorbate-80 etc.) and water soluble organic solvents (e.g., MPD, acetonitrile, alcohols, diols etc.).

As will be understood by one of skill in the art, the conditions under which any of the salts identified above may be incorporated into the presently disclosed HIC step may be determined empirically without undue experimentation. For example, the Hofmeister series provides a classification of ions based on their ability to “salt out” (i.e., decrease solubility; precipitate) or “salt in” (i.e., increase solubility; solubilize) proteins. The first salts of the Hofmeister series strengthen hydrophobic interactions and increase solvent surface tension such that non-polar molecules “salt out.” Later salts of the Hofmeister series weaken hydrophobic interactions and increase solubility such that non-polar molecules “salt in.” The salt(s) that may be effective for a given HIC resin and/or target protein may first be identified by reference to the Hofmeister series. Selected salt(s) may then be titrated on the HIC resin to more precisely delineate the binding, washing and elution kinetics of the target protein. For example, 2M NaCl has been identified as a beneficial salt concentration for HIC purification of BMPs.

In one embodiment, the first step may include testing salts in the Hofmeister series for effectiveness in promoting binding and identifying concentrations (e.g., Molarity or grams/L) of those salts at which BMP completely binds to the HIC medium. Similarly, a second step may include determining the concentrations of the Hofmeister series salts at which BMP does not bind to the HIC medium. A third step may then include choosing the particular Hofmeister series salt and identifying the concentration of that particular salt at which BMP starts to elute by flushing a BMP-loaded HIC medium with successive aliquots of buffer, each aliquot having an incrementally lower salt concentration than the previous aliquot. Starting at the salt concentration at which BMP is known to bind the HIC medium (i.e., 2.0 M NaCl) and incrementally approaching the salt concentration at which BMP is known to not bind the HIC medium (i.e., 1.0M NaCl) in 0.1M increments will identify the salt concentration at which BMP elutes from the HIC medium (e.g., BMP elutes at approximately 1.4-1.5M NaCl). Alternatively a descending salt gradient may be utilized, starting at the salt concentration at which BMP is known to bind the HIC medium and decreasing the concentration to below the salt concentration at which BMP is known to not bind the HIC medium.

### Cation Exchange Polishing (Step-3)

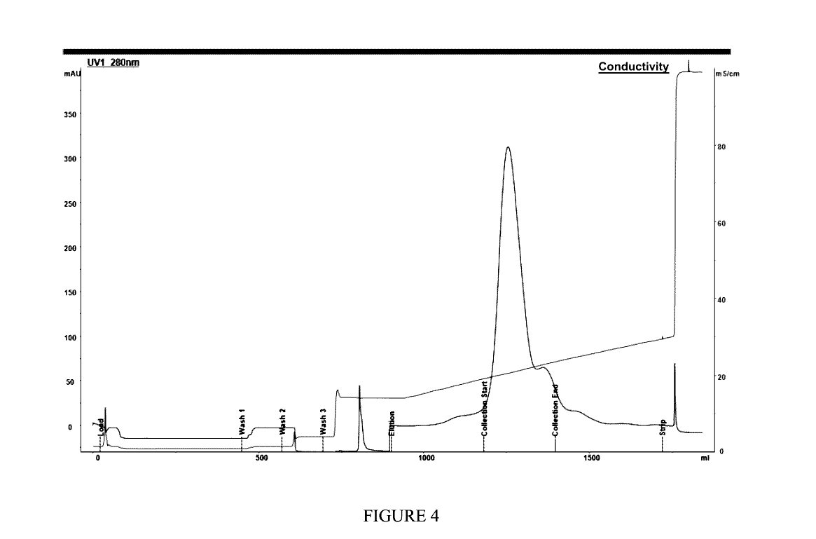

Following the phenyl 6FF HIC process described above, a cation exchange chromatography medium is preferably used to remove MPD or other solubilizing agents, further clear the eluent of DNA, host-cell proteins, endotoxins and other non-proteinaceous contaminants and concentrate the BMP. Referring to FIG. 4, in an exemplary cation exchange polishing step, the eluent from Phenyl 6FF HIC medium may be loaded onto a Capto™ S Impact (GE Life Sciences, Marlborough, Mass.) cation exchange chromatography medium and then washed to remove impurities. The first wash may include one volume of 50 mM glycine, pH 3.0; the second wash may include 50 mM glycine, 6M urea, pH 3.0; and the third wash may include 50 mM Tris, 6M urea, 0.15M NaCl, pH 7.0. Gradient elution of the medium may then be performed using

Page 14 50 mM Tris, 6M urea, 0.4M NaCl, pH 7.0 (20 CV). The BMP yield following the third column step is 70-99%, with a purity verified by reverse phase chromatography of 93-99%.

### Ultrafiltration/Diafiltration

An additional step in the purification process of the present disclosure may include an ultrafiltration/diafiltration (UF/DF) step in which tangential flow filtration system is used for buffer exchange and concentration of the BMP. A filter membrane device that includes a specific molecular weight cut-off may be used to retain large molecular weight proteins (e.g., BMP) while lower molecular weight components are removed. By continuously adding new buffer to the retentate at the same rate that permeate solution is flowing out filter membrane, the original buffer components are gradually diluted away. For example, the eluent from the cation exchange step may be loaded onto a 10 kDa Hydrosart® ultrafiltration membrane (Sartorius, AG) and diafiltered with 5-7 diafiltration volumes of 50 mM Glycine, 6M Urea, pH 3.0 until >99% buffer exchange into 50 mM Glycine, 6M Urea, pH 3.0 has been achieved. This may be subsequently followed by a further buffer exchange into 50 mM acetic acid using a similar procedure as the first buffer exchange. Once the initial buffer exchange phase is complete, the BMP is typically concentrated approximately 5-fold to achieve a concentration of 4-6 mg/mL BMP. Once the protein has been concentrated, a second buffer exchange is performed to place the BMP in its final formulation buffer (e.g., 25 mM glutamic acid, 2% glycine, 1% sucrose, pH 4.0). This is accomplished by diafiltering with an additional 7-9 diafiltration volumes of formulation buffer, after which the retentate is drained and residual protein recovered by flushing the system with 0.5-1 hold-up volumes of formulation buffer. Surprisingly, the BMP yield following the UF/DF step is 90-98%.

In one embodiment, the UF/DF step may optionally be preceded by a viral filtration step. For example, the eluent from the cation exchange step may be loaded onto a Viresolve® Pro Solution (EMD Millipore) viral filtration device, rinsed with water for pharmaceutical use (WPU), equilibrated with 3% v/v acetic acid buffer and filtered under constant flow. The BMP yield following the viral filtration step is 95-100%, with a purity verified by reverse phase chromatography of 92-99%.

Following the UF/DF step, the concentrated BMP may undergo a final filtration step using, for example, a Sartopore 2® PES filter unit under constant flow. The BMP yield following this final filtration step is 95-100%.

Although the embodiments and exemplary data of present disclosure make specific reference to methods for purification of BMP, it should be appreciated that the present disclosure may be applied with equivalent or similar results to other members of the TGF-β superfamily that exhibit similar solubility and/or self-aggregation characteristics. Examples of BMPs that may be amenable to purification using the methods disclosed herein include, but are in no way limited to, BMP-2, BMP-3, BMP-4, BMP-5, BMP-6, BMP-7, BMP-9, BMP-12, BMP-13 and recombinant, homodimeric, heterodimeric, mutant and/or chimeric versions thereof.

The phrase “and/or,” as used herein should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “consists essentially of” means excluding other materials that contribute to function, unless otherwise defined herein. Nonetheless, such other materials may be present, collectively or individually, in trace amounts.

As used in this specification, the term “substantially” or “approximately” means plus or minus 10% (e.g., by weight or by volume), and in some embodiments, plus or minus 5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

Certain embodiments of the present invention have been described above. It is, however, expressly noted that the present invention is not limited to those embodiments, but rather the intention is that additions and modifications to what was expressly described herein are also included within the scope of the invention. Moreover, it is to be understood that the features of the various embodiments described herein are not mutually exclusive and can exist in various combinations and permutations, even if such combinations or permutations are not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description.

What is claimed is:

1. A method of purifying a bone morphogenetic protein (BMP) from a fluid, comprising the steps of:
contacting the fluid comprising the BMP with a hydrophobic interaction chromatography medium under conditions in which the BMP is soluble within the fluid, wherein the fluid includes at least one salt at a concentration above a predetermined threshold, thereby facilitating an association of the BMP with the hydrophobic interaction chromatography medium;
contacting the hydrophobic interaction chromatography medium with a first mobile phase comprising a first agent that promotes the solubility of the BMP, the first mobile phase having a salt concentration similar to a salt concentration of the initial fluid;
contacting the hydrophobic interaction chromatography medium with a second mobile phase lacking the first agent that promotes the solubility of the BMP, thereby increasing an association between the BMP with the chromatography medium;
contacting the hydrophobic interaction chromatography medium with a third mobile phase having a dissimilar salt concentration relative to one or more of the fluid, the first mobile phase and the second mobile phase, thereby decreasing an association of a second, non-BMP with the hydrophobic interaction chromatography medium; and
eluting the BMP from the hydrophobic interaction chromatography medium by contacting the hydrophobic interaction chromatography medium with an elution mobile phase comprising a second agent, different from the first agent, that promotes the solubility of the BMP and disrupts the association with the hydrophobic interaction chromatography media.

2. The method of claim 1, wherein a concentration of the second agent is varied over time.

3. The method of claim 1, wherein a concentration of the second agent is constant over time.

4. The method according to claim 1, wherein the hydrophobic interaction chromatography medium is not functionalized with a peptide affinity ligand.

5. The method according to claim 1, wherein the first agent that promotes the solubility of the BMP is urea.

6. The method according to claim 5, wherein the urea is present in the first mobile phase at a concentration of 5-8M.

7. The method according to claim 1, wherein the first mobile phase includes 50 mM glycine and 2M sodium chloride.

8. The method according to claim 5, wherein urea is present in the fluid at a concentration of at least 3M.

9. The method according to claim 8, wherein the fluid includes at least 1M sodium chloride.

10. The method according to claim 1, wherein the second agent that promotes the solubility of the BMP is hexylene glycol.

11. The method according to claim 1, wherein the fluid includes an eluent from an ion exchange chromatography medium.

12. The method according to claim 1, wherein a product BMP yield is at least 60%.

13. The method according to claim 1, wherein a purity of the BMP is at least 90%.

14. A method of purifying a bone morphogenetic protein (BMP) from a sample, comprising the steps of:
loading an affinity-like chromatography medium with a solution containing BMP under conditions such that at least a portion of the BMP binds to the affinity-like chromatography medium;
eluting at least a portion of the BMP from the affinity-like chromatography medium;
loading a hydrophobic interaction chromatography medium with the BMP-containing eluent from affinity-like chromatography medium under conditions such that at least a portion of the BMP binds to the hydrophobic interaction chromatography medium;
eluting at least a portion of the BMP from the hydrophobic interaction chromatography medium;
loading a cation exchange medium with the BMP-containing eluent from the hydrophobic interaction chromatography medium under conditions such that at least a portion of the BMP binds to the cation exchange medium;
eluting at least a portion of the BMP from the cation exchange medium; and
concentrating the BMP in a suitable buffer.

15. A method of purifying a bone morphogenetic protein (BMP) from a fluid, comprising the steps of:
loading the fluid containing BMP onto a hydrophobic interaction medium, wherein the fluid includes urea and a first salt at a first concentration, and wherein the BMP is in solution in the fluid;
washing the hydrophobic interaction medium with a first solution, wherein a concentration of the salt in the first solution is less than the first concentration, the first solution does not include urea, and the BMP is less soluble in the first solution than in the fluid; and
eluting the BMP with a second solution that does not include the first salt or urea.

16. The method according to claim 15, wherein the urea is present in the fluid at a concentration of at least 3M.

17. The method according to claim 15, wherein the fluid includes at least 1M sodium chloride.

18. The method according to claim 15, wherein the second solution promotes the solubility of the BMP.

19. The method according to claim 18, wherein the second solution includes hexylene glycol.

20. A method of purifying a bone morphogenetic protein (BMP) from a first solution, comprising the steps of:
contacting a hydrophobic interaction chromatography medium with the first solution, wherein the first solution is characterized by a first solubility of the BMP therein;
contacting the hydrophobic interaction chromatography medium with a second solution characterized by a second solubility of the BMP that is less than the first solubility; and
contacting the hydrophobic interaction chromatography medium with a third solution characterized by a third solubility of the BMP that is greater than the second solubility.
